(19) United States
(12) Patent Application Publication
(10) Pub. No.: US 2025/0097654 A1
(43) Pub. Date: Mar. 20, 2025

(54) HEARING DEVICE

(71) Applicant: Starkey Laboratories, Inc., Eden Prairie, MN (US)

(72) Inventors: Sidney A. Higgins, Maple Grove, MN (US); David Tourtelotte, Eden Prairie, MN (US); Craig C. Feldsien, Prior Lake, MN (US)

(21) Appl. No.: 18/369,865

(22) Filed: Sep. 19, 2023

(51) Int. Cl. H04R 25/00; A61B 5/00; A61B 5/024; G10K 11/178

(52) U.S. Cl. CPC H04R 25/656; A61B 5/02438; A61B 5/6817; G10K 11/17823; G10K 11/17873; H04R 25/505; H04R 25/604; H04R 25/609; H04R 25/70; G10K 2210/1081; G10K 2210/3027; H04R 2225/0216; H04R 2460/01

(57) ABSTRACT

Various embodiments of a hearing device and a system including such device are disclosed. The device includes an enclosure having a front housing and a rear housing. The enclosure further includes an isolator disposed between the front housing and the rear housing. The isolator includes a body, a first sleeve disposed at the first end of the body, and a second sleeve disposed at the second end of the body. A second end of the front housing is connected to the first sleeve of the isolator and a second end of the rear housing is connected to the second sleeve of the isolator. The hearing device further includes a first sensor disposed in the front housing, a second sensor disposed in the rear housing, and a receiver disposed at least partially within the body of the isolator.

### BACKGROUND

Hearing devices that are disposed in an ear of a wearer or inserted into an opening of an ear canal of the wearer typically include a housing or shell with electronic components such as a receiver (i.e., speaker) disposed within the housing. The receiver is adapted to provide acoustic information in the form of acoustic energy to the wearer's ear canal from a controller either disposed within the housing of the hearing device or connected to the hearing device by a wired or wireless connection. This acoustic information can include music or speech from a recording or other source. In hearing devices such as hearing assistance devices, the acoustic information provided to the wearer can include ambient sounds such as speech from a person or persons that are speaking in proximity to the wearer. Such speech can be amplified so that the wearer can better hear the speaker. Some hearing devices also include a microphone disposed within the housing. The microphone can be utilized to detect the wearer's voice and provide a microphone signal to the receiver, which in turn provides acoustic energy to the ear that includes an amplified version of the wearer's voice.

### SUMMARY

In general, the present disclosure provides various embodiments of a hearing device and a system that includes such device. The hearing device can include an enclosure having a front housing and a rear housing. The enclosure further includes an isolator disposed between the front and rear housings. The isolator can include a body, a first sleeve disposed at a first end of the body and a second sleeve disposed at a second end of the body. The first sleeve can be connected to the front housing, and the second sleeve can be connected to the rear housing. The device can further include a first sensor disposed in the front housing, a second sensor disposed in the rear housing, and a receiver disposed at least partially within the body of the isolator. In one or more embodiments, the isolator can be adapted to reduce vibrations caused by the receiver that can affect a signal produced by at least one of the first sensor or second sensor. Further, at least one of the first sensor or second sensor can include a microphone.

In one aspect, the present disclosure provides a hearing device that includes an enclosure extending along an enclosure axis. The enclosure includes a front housing that extends along the enclosure axis between a first end and a second end and includes an opening disposed in the first end, a rear housing that extends along the enclosure axis between a first end and a second end, and an isolator disposed between the front housing and the rear housing. The isolator includes a body that extends along the enclosure axis between a first end and a second end, a first sleeve disposed at the first end of the body, and a second sleeve disposed at the second end of the body. The second end of the front housing is connected to the first sleeve of the isolator, and the second end of the rear housing is connected to the second sleeve of the isolator. The hearing device further includes a first sensor disposed in the front housing, a second sensor disposed in the rear housing, and a receiver disposed at least partially within the body of the isolator.

In another aspect, the present disclosure provides a hearing device system that includes a hearing device, a hearing module, and a cable that connects the hearing device to the hearing module. The hearing device includes an enclosure extending along an enclosure axis. The enclosure includes a front housing that extends along the enclosure axis between a first end and a second end and includes an opening disposed in the first end, a rear housing that extends along the enclosure axis between a first end and a second end, and an isolator disposed between the front housing and the rear housing. The isolator includes a body that extends along the enclosure axis between a first end and a second end, a first sleeve disposed at the first end of the body, and a second sleeve disposed at the second end of the body. The second end of the front housing is connected to the first sleeve of the isolator, and the second end of the rear housing is connected to the second sleeve of the isolator. The hearing device further includes a first sensor disposed in the front housing, a second sensor disposed in the rear housing, and a receiver disposed at least partially within the body of the isolator. Further, the hearing module is adapted to be disposed between an ear and a skull of a wearer, where the hearing module includes a module housing and electronic components disposed within the module housing.

In another aspect, the present disclosure provides a method that includes forming a body of an isolator, disposing a first sleeve at a first end of the body, and disposing a second sleeve at a second end of the body. The method further includes disposing a first sensor within a front housing, where the front housing includes a first end, a second end, and an opening disposed in the first end; disposing a second sensor within a rear housing, where the rear housing includes a first end and a second end; and disposing a receiver at least partially within the body of the isolator. The method further includes connecting the second end of the front housing to the first sleeve; and connecting the second end of the rear housing to the second sleeve, where the front housing, the isolator, and the rear housing define an enclosure that extends along an enclosure axis.

All headings provided herein are for the convenience of the reader and should not be used to limit the meaning of any text that follows the heading, unless so specified.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements. The term “consisting of” means “including,” and is limited to whatever follows the phrase “consisting of.” Thus, the phrase “consisting of” indicates that the listed elements are required or mandatory and that no other elements may be present. The term “consisting essentially of” means including any elements listed after the phrase and is limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

In this application, terms such as “a,” “an,” and “the” are not intended to refer to only a singular entity but include the general class of which a specific example may be used for having Illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

These and other aspects of the present disclosure will be apparent from the detailed description below. In no event, however, should the above summaries be construed as limitations on the claimed subject matter, which subject matter is defined solely by the attached claims, as may be amended during prosecution.

### BRIEF DESCRIPTION OF THE DRAWINGS

Throughout the specification, reference is made to the appended drawings, where like reference numerals designate like elements, and wherein:

FIG. 1 is a schematic perspective view of one embodiment of a hearing device system that includes a hearing device and a hearing module connected to the hearing device by a cable.

FIG. 2 is a schematic perspective view of the hearing device of FIG. 1.

FIG. 3 is a schematic cross-section view of the hearing device of FIG. 1.

FIG. 4 is a side perspective view of a front housing of an enclosure of the hearing device of FIG. 1.

FIG. 5 is a schematic rear perspective view of the front housing of FIG. 4.

FIG. 6 is a schematic perspective view of an isolator of the hearing device of FIG. 1.

FIG. 7 is an exploded view of the isolator of FIG. 6.

FIG. 8 is a schematic diagram of the hearing device system of FIG. 1.

FIG. 9 is a schematic cross-section view of another embodiment of a hearing device that can be utilized with the hearing device system of FIG. 1.

FIG. 10 is a flowchart of a method of forming the hearing device of FIG. 1.

### DETAILED DESCRIPTION

In general, the present disclosure provides various embodiments of a hearing device and a system that includes such device. The hearing device can include an enclosure having a front housing and a rear housing. The enclosure further includes an isolator disposed between the front and rear housings. The isolator can include a body, a first sleeve disposed at a first end of the body and a second sleeve disposed at a second end of the body. The front housing can be connected to the front housing, and the second sleeve can be connected to the rear housing. The device can further include a first sensor disposed in the front housing, a second sensor disposed in the rear housing, and a receiver disposed at least partially within the body of the isolator. In one or more embodiments, the isolator can be adapted to reduce vibrations caused by the receiver that can affect a signal produced by at least one of the first sensor or second sensor. Further, at least one of the first sensor or second sensor can include a microphone.

Currently available in-ear hearing devices that include a microphone disposed within a housing of the device and directed toward an ear canal of the wearer have the microphone disposed in a planar manner on top of a receiver also disposed within the housing. Such configuration can, however, require larger custom earbuds that can make it difficult to fit ear canals of various wearers. Further, vibrations from the receiver can be picked up by the microphone, thereby reducing a signal to noise ratio of the microphone signal. These receiver vibrations can either be directly from the receiver and/or from the housing that vibrates in response to the receiver.

One or more embodiments of hearing devices described herein can exhibit various advantages over known hearing devices. For example, the isolator that is disposed between the front and rear housings can reduce vibrations from the receiver and the enclosure. Such vibration reduction can enable placement of the first sensor such as a microphone inside the front housing without an increase in noise in the microphone signal that can be caused by vibration of the receiver. Further, such reduced vibrations can also enable placement of the second sensor such as a second microphone inside the rear housing without an increase in noise in the microphone signal of the second microphone.

The isolator can include the body, the first sleeve disposed at the first end of the body, and the second sleeve disposed at the second end of the body. In one or more embodiments, at least one of the first or second sleeves can be over-molded onto the body, which can enhance robustness of the isolator and the enclosure of the device and increase manufacturability of the device. Further, additional functionalities of the hearing device can be enhanced by this reduction of receiver vibration as the first sensor can be placed forward of the receiver and thus further into the ear canal. For example, circuitry disposed within the hearing device or connected to the device by a wired or wireless connection can perform, e.g., active noise cancellation, self-fit testing, heartbeat monitoring, heartbeat variability monitoring, occlusion suppression, respiration rate monitoring, wheezing detection/monitoring, etc., based upon acoustic information provided by the first sensor. Further, the first sensor can be utilized to detect otoacoustic emissions, disorders of the canal and tympanic membrane such as partially or fully wax-blocked ear canal, damage to the tympanic membrane, or canal tissue inflammation. In one or more embodiments, wind noise can be detected at both the first sensor and second sensor, and a controller of the hearing device system can be utilized to alternate between the first and second sensors to enhance performance of the system by selecting the sensor that is experiencing the lowest level of wind noise. In embodiments where the first and second sensors are microphones, a third microphone disposed on or in a housing of a hearing module of the system can provide a three-microphone system that can provide enhanced directional performance. Further, one or both of the first and second sensors can be utilized to assist the wearer in finding the hearing device.

In one or more embodiments where at least one of the first or second sensors are microphones, a microphone disposed on or within the module housing of the hearing module may no longer be required, thereby eliminating microphone ports in the housing. With a sealed cable port, the module housing would not have any access points for ingress of moisture and/or debris, thereby potentially providing a waterproof module housing. For example, the hearing device can include a microphone disposed in the rear housing of the device such that it is outward facing. Such outward facing microphone may eliminate the need for a microphone disposed on or within the hearing module of the system and thus can serve as the system's sole microphone. Further, directionality of sound waves that are detected by the outward facing microphone can be determined based upon pinna cues.

Embodiments of the disclosure are defined in the claims; however, herein there is provided a non-exhaustive listing of non-limiting examples. Any one or more of the features of these examples may be combined with any one or more features of another example, embodiment, or aspect described herein.

Example Ex1. A hearing device that includes an enclosure extending along an enclosure axis. The enclosure includes a front housing that extends along the enclosure axis between a first end and a second end and includes an opening disposed in the first end, a rear housing that extends along the enclosure axis between a first end and a second end, and an isolator disposed between the front housing and the rear housing. The isolator includes a body that extends along the enclosure axis between a first end and a second end, a first sleeve disposed at the first end of the body, and a second sleeve disposed at the second end of the body. The second end of the front housing is connected to the first sleeve of the isolator, and the second end of the rear housing is connected to the second sleeve of the isolator. The hearing device further includes a first sensor disposed in the front housing, a second sensor disposed in the rear housing, and a receiver disposed at least partially within the body of the isolator.

Example Ex2. The device of Ex1, further including an acoustic port that extends through the first end of the isolator body between the receiver and the opening disposed in the first end of the front housing, where the acoustic port acoustically connects the receiver to the opening.

Example Ex3. The device of any one of Ex1-Ex2, further including a first sensor port that extends between the first sensor and the opening disposed in the first end of the front housing, where the first sensor port operatively connects the first sensor to the opening.

Example Ex4. The device of Ex3, where an inlet of the first sensor port defines a plane that is orthogonal to the enclosure axis, where a distance between an outlet of the acoustic port and the plane is no greater than 1.2 mm as measured in a direction parallel to the enclosure axis.

Example Ex5. The device of Ex3, where the first sensor further includes a manifold that extends along the enclosure axis and defines a portion of the first sensor port.

Example Ex6. The device of Ex3, further including a second sensor port that extends between the second sensor and an opening disposed adjacent to the first end of the rear housing, where the second sensor port operatively connects the second sensor to the opening.

Example Ex7. The device of any one of Ex1-Ex6, where the isolator body includes a ledge disposed proximate to the first end, where the first sleeve is disposed on the ledge.

Example Ex8. The device of any one of Ex1-Ex7, where the isolator body includes an elastomeric material.

Example Ex9. The device of any one of Ex1-Ex8, where the first sleeve of the isolator includes a rib that extends in a direction away from the first end of the body, where the rib is adapted to be inserted into the second end of the front housing.

Example Ex10. The device of Ex9, where the first sleeve of the isolator further includes a ledge that extends from the rib to a perimeter of the first sleeve, where an end surface of the second end of the front housing is adapted to engage the ledge.

Example Ex11. The device of any one of Ex1-Ex10, where the second sleeve of the isolator further includes a rib that extends in a direction away from the second end of the body, where the rib is adapted to be inserted into the second end of the rear housing.

Example Ex12. The device of Ex11, where the second sleeve of the isolator further includes a ledge that extends from the rib to a perimeter of the second sleeve, where an end surface of the second end of the rear housing is adapted to engage the ledge.

Example Ex13. The device of any one of Ex1-Ex12, where at least one of the first sensor or the second sensor includes a microphone.

Example Ex14. A hearing device system that includes a hearing device, a hearing module, and a cable that connects the hearing device to the hearing module. The hearing device includes an enclosure extending along an enclosure axis. The enclosure includes a front housing that extends along the enclosure axis between a first end and a second end and includes an opening disposed in the first end, a rear housing that extends along the enclosure axis between a first end and a second end, and an isolator disposed between the front housing and the rear housing. The isolator includes a body that extends along the enclosure axis between a first end and a second end, a first sleeve disposed at the first end of the body, and a second sleeve disposed at the second end of the body. The second end of the front housing is connected to the first sleeve of the isolator, and the second end of the rear housing is connected to the second sleeve of the isolator. The hearing device further includes a first sensor disposed in the front housing, a second sensor disposed in the rear housing, and a receiver disposed at least partially within the body of the isolator. Further, the hearing module is adapted to be disposed between an ear and a skull of a wearer, where the hearing module includes a module housing and electronic components disposed within the module housing.

Example Ex15. The system of Ex14, where the electronic components of the hearing module include a controller that is operatively connected to the hearing device.

Example Ex16. The system of Ex15, where the controller is adapted to direct a noise canceling signal to the receiver of the hearing device that is based upon a noise signal received from the first sensor of the hearing device, where the receiver is adapted to direct a noise canceling acoustic wave into an ear canal of the wearer of the hearing device that is based upon the noise canceling signal from the controller.

Example Ex17. The system of Ex15, where the controller is adapted to determine a fit of the hearing device in an ear canal of the wearer based upon a feedback signal from the first sensor of the hearing device in response to a fit-test acoustic wave directed into the ear canal by the receiver.

Example Ex18. The system of Ex15, where the controller is adapted to measure a pulse rate of the wearer based upon a pulse signal received from the first sensor of the hearing device, where the pulse signal is based upon a pulse detected by the first sensor.

Example Ex19. The system of Ex15, where the controller is adapted to measure an occlusion value of the hearing device in an ear canal of the wearer based upon an occlusion signal received from the first sensor of the hearing device in response to an acoustic wave directed into the ear canal by the receiver and detected by the first sensor.

Example Ex20. The system of any one of Ex14-Ex19, where the electronic components of the hearing module include a microphone, where each of the first sensor and second sensor includes a microphone.

Example Ex21. A method that includes forming a body of an isolator, disposing a first sleeve at a first end of the body, and disposing a second sleeve at a second end of the body. The method further includes disposing a first sensor within a front housing, where the front housing includes a first end, a second end, and an opening disposed in the first end; disposing a second sensor within a rear housing, where the rear housing includes a first end and a second end; and disposing a receiver at least partially within the body of the isolator. The method further includes connecting the second end of the front housing to the first sleeve; and connecting the second end of the rear housing to the second sleeve, where the front housing, the isolator, and the rear housing define an enclosure that extends along an enclosure axis.

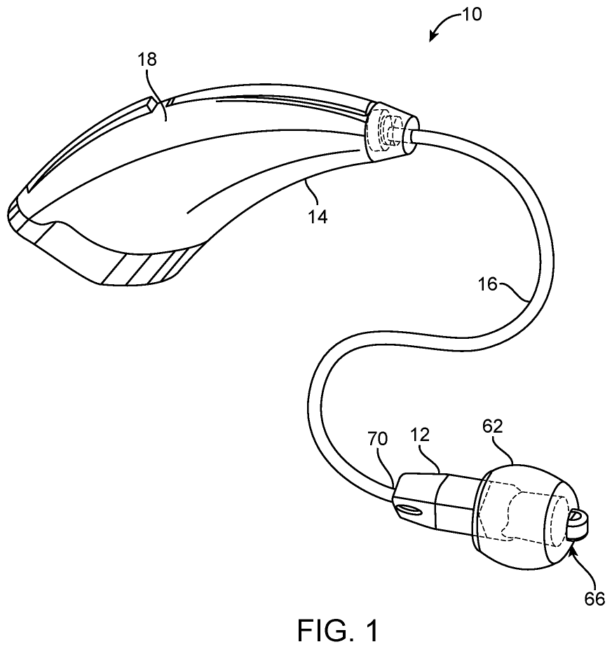

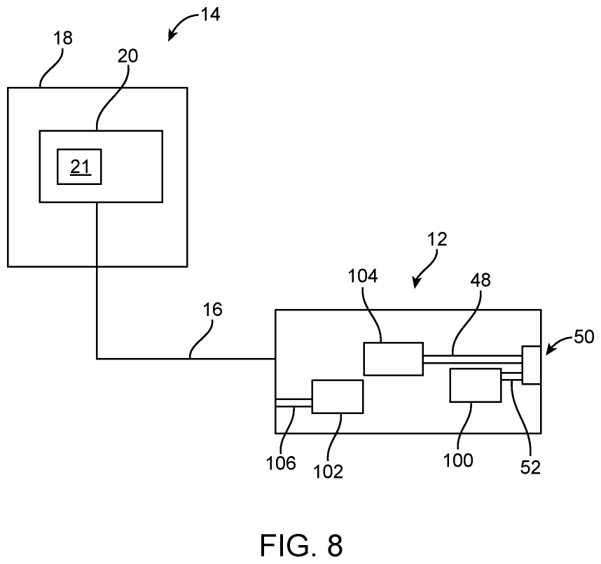

FIG. 1 is a schematic perspective view of one embodiment of a hearing device system 10. The system 10 includes a hearing device 12, a hearing module 14, and a cable 16 that connects the hearing device to the hearing module. The hearing module 14 is adapted to be disposed between an ear and a skull of a wearer. As is further described herein, the hearing module 14 includes a module housing 18 and electronic components (electronic components 20 of FIG. 8) disposed within the module housing.

The hearing device 12 can include any suitable device that can provide acoustic energy to a wearer using any suitable technique or techniques, e.g., by directing sound into the ear of the wearer, bone conduction, implants, etc. See, e.g., one or more embodiments of hearing devices described in U.S. patent application Ser. No. 18/121,317, field Mar. 14, 2023, and entitled HEARING DEVICE; and U.S. patent application Ser. No. 18/121,325, field Mar. 14, 2023, and entitled HEARING DEVICE. In one or more embodiments, the hearing device 12 can include over-the-ear or in-ear headphones, an earpiece, etc. Further, in one or more embodiments, the system 10 can include a hearing assistance device such as behind-the-ear (BTE), in-the-ear (ITE), in-the-canal (ITC), or completely-in-the-canal (CIC) type hearing devices. It is understood that behind-the-ear type hearing devices can reside substantially behind the ear or over the ear. Such devices can include receivers associated with an electronics portion of the behind-the-ear device, or receivers disposed in the ear canal of the user. Such devices are also known as receiver-in-the-canal (RIC) or receiver-in-the-ear (RITE) hearing devices.

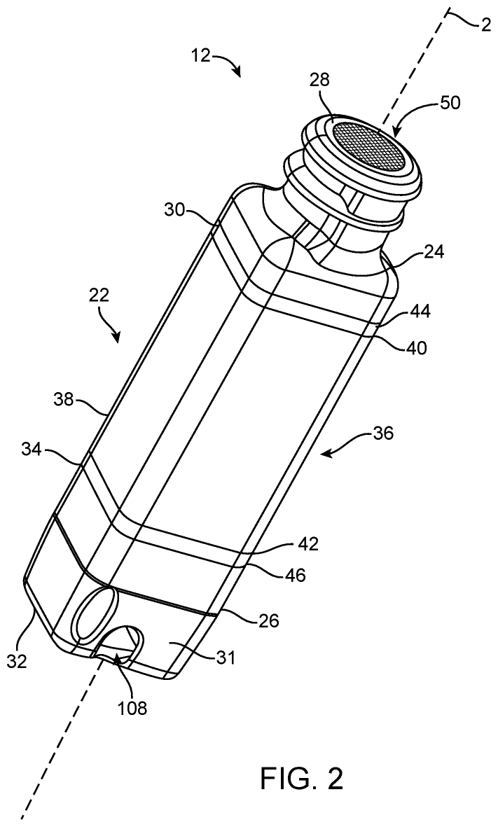

As shown in FIGS. 2-8, the hearing device 12 includes an enclosure 22 that extends along an enclosure axis 2. The enclosure 22 includes a front housing 24 and a rear housing 26. The front housing 24 extends along the enclosure axis 2 between a first end 28 and a second end 30. Further, the rear housing 26 extends along the enclosure axis 2 between a first end 32 and a second end 34. The front housing 24 includes an opening 50 disposed in the first end 28.

The enclosure 22 of the device 12 also includes an isolator 36 disposed between the front housing 24 and the rear housing 26. The isolator 36 includes a body 38 that extends along the enclosure axis 2 between a first end 40 and a second end 42. The isolator 36 further includes a first sleeve 44 disposed at the first end 40 of the body 38 and a second sleeve 46 disposed at the second end 42 of the body. The second end 30 of the front housing 24 is connected to the first sleeve 44 of the isolator 36, and the second end 34 of the rear housing 26 is connected to the second sleeve 46 of the isolator.

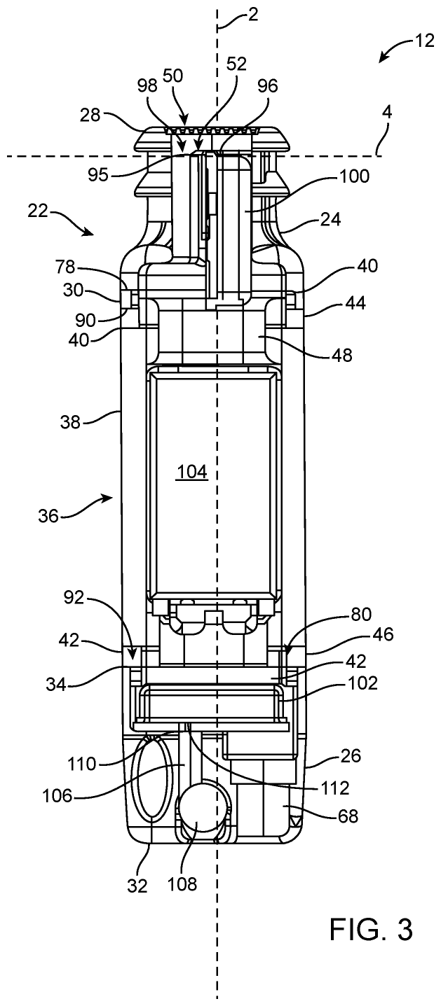

Any suitable electronic components can be disposed within the enclosure 22. As shown in FIG. 3, the hearing device 12 includes a first sensor 100 disposed in the front housing 24, a second sensor 102 disposed in the rear housing 26, and a receiver 104 disposed at least partially within the body 38 of the isolator 36. In one or more embodiments, at least one of the first sensor 100 or second sensor 102 includes a microphone (referred to herein as first microphone 100 and/or second microphone 102). Although depicted as including the first and second sensors 100, 102 and the receiver 104, in one or more embodiments, one or more additional components and/or circuitry can be disposed at least partially within or on the enclosure 22, e.g., at least one of a sensor, controller, amplifier, filter, Giant Magneto Resistance sensor, switch, or outward facing microphone.

The device 12 further includes an acoustic port 48 that extends through the first end 40 of the isolator body 38 between the receiver 104 and the opening 50 disposed in the first end 28 of the front housing 24. The acoustic port 48 acoustically connects the receiver 104 to the opening 50, i.e., the acoustic port is adapted to direct acoustic energy between the receiver and the opening.

Further, the device 12 includes a first sensor port 52 that extends between the first sensor 100 and the opening 50 disposed in the first end 28 of the front housing 24. In one or more embodiments, the first sensor port 52 can operatively connect the first sensor 100 to the opening 50. For example, in one or more embodiments, the first sensor port 52 can acoustically connect the first sensor 100 to the opening 50. Further, the device 12 includes a second sensor port 106 that extends between an inlet 112 of the second sensor 102 and an opening 108 disposed adjacent to the first end 32 of the rear housing 26. The second sensor port 106 operatively connects the second sensor 102 to the opening 108. For example, in one or more embodiments, the second sensor port 106 acoustically connects the second sensor 102 to the opening 108.

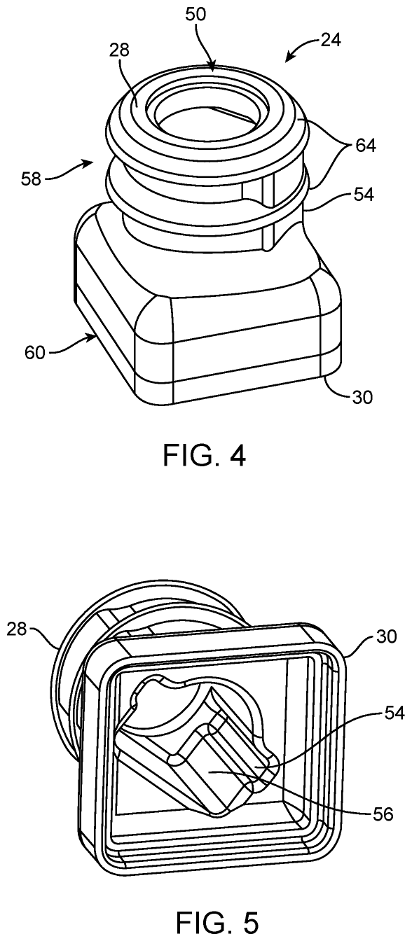

The front housing 24 of the enclosure 22 extends along the enclosure axis 2 between the first end 28 and the second end 30. The front housing 24 can take any suitable shape and have any suitable dimensions. In one or more embodiments, the front housing 24 is sized such that at least a portion of its first end 28 can be disposed within an opening of the ear canal of the wearer. As shown in FIG. 4, the front housing 24 can include a first portion 58 and a second portion 60. The first portion 58 can be adapted to be at least partially disposed within the opening of the ear canal, and the second portion 60 can be adapted to be connected to the isolator 36.

In one or more embodiments, the hearing device 12 can include an earbud 62 (FIG. 1) connected to the first end 28 of the front housing 24. The earbud 62 can take any suitable shape and having any suitable dimensions. In one or more embodiments, the earbud 62 is integral with the front housing 24, i.e., formed as a single part with the front housing during the manufacturing process. In one or more embodiments, the earbud 62 can be manufactured separately from the front housing 24 and connected to the front housing using any suitable technique. The earbud 62 includes an opening 66 that is aligned along the enclosure axis 2 with the opening 50 in the first end 28 of the front housing 24.

The front housing 24 can include one or more flanges 64 (FIG. 4) that are adapted to retain the earbud 62. Further, each flange 64 can take any suitable shape and have any suitable dimensions. In one or more embodiments, at least one of the flanges 64 can be a concentric flange.

As shown in FIGS. 4-5, the front housing 24 can also include a slot 54 that is disposed in an inner surface 56 of the front housing. The slot 54 is adapted to receive the first sensor 100 and can take any suitable shape and have any suitable dimensions. In one or more embodiments, the slot 54 extends in a direction parallel to the enclosure axis 2.

The front housing 24 can include any suitable material, e.g., at least one of a polymeric material, metallic material, or non-metallic inorganic material. Suitable polymeric materials include thermoplastic polymers (e.g., thermoplastic polyurethanes, thermoplastic elastomers), thermoset polymers, photopolymers, etc. In one or more embodiments, the front housing 24 can include the same material as the rear housing 26. Further, in one or more embodiments, the front housing 24 can include the same material as the material of the body 38 of the isolator 36. Further, the front housing 24 can be manufactured utilizing any suitable technique, e.g., molding, injection molding, 3D printing, die-casting, metal injection molding, sintering, stamping, casting, etc.

As mentioned herein, the rear housing 26 of the enclosure 22 extends along the enclosure axis 2 between the first end 32 and the second end 34. The rear housing 26 can take any suitable shape have any suitable dimensions. Further, the rear housing 26 can include any suitable material, e.g., at least one of a polymeric material, metallic material, or non-metallic inorganic material. Suitable polymeric materials include thermoplastic polymers (e.g., thermoplastic polyurethanes, thermoplastic elastomers), thermoset polymers, photopolymers, etc. In one or more embodiments, the rear housing 26 can include the same material as the front housing 24. Further, in one or more embodiments, the rear housing 26 can include the same material as the material of the body 38 of the isolator 36. Further, the rear housing 26 can be manufactured utilizing any suitable technique, e.g., molding, injection molding, 3D printing, die-casting, metal injection molding, sintering, stamping, casting, etc.

The device 12 can include a connector port 68 (FIG. 3) disposed in the first end 32 of the rear housing 26 that extends between the first end and the second sensor 102 disposed within the rear housing. The connector port 68 can be adapted to receive an end 70 of the cable 16 (FIG. 1) that connects the hearing device 12 to the hearing module 14 such that the cable can connect the first and second sensors 100, 102, the receiver 104, and any other circuitry disposed on or within the enclosure 22 to circuitry 20 disposed within the hearing module 14.

The front housing 24 and the rear housing 26 can be connected using any suitable technique to provide the enclosure 22. For example, the isolator 36 can be disposed between the front housing 24 and the rear housing 26, where the front housing and the rear housing are connected to the isolator. The second end 30 of the front housing 24 and the second end 34 of the rear housing 26 are connected to the isolator 36 using any suitable technique, e.g., bonding, adhering including adhesive bonding and adhesive tapes, welding, friction-fitting, snap fitting, etc. The front and rear housings 24, 26 can be connected to any suitable portion or portions of the isolator 36. In one or more embodiments, one or both of the front housing 24 and rear housing 26 can be removably connected to the isolator 36 such that front and rear housings can be replaced. Such removable connection between the front and rear housings 24, 26 and the isolator 36 can provide a modular hearing device 12.

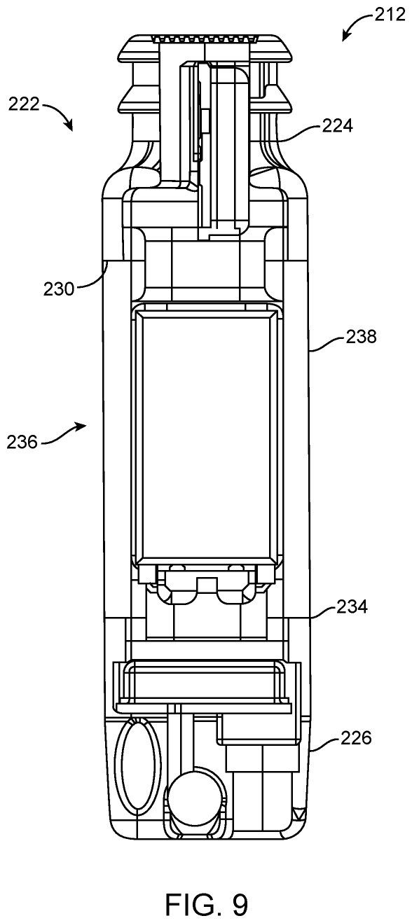

The isolator 36 includes the body 38, the first sleeve 44 disposed at the first end 40 of the body, and the second sleeve 46 disposed at the second end 42 of the body. Although depicted as including first and second sleeves 44, 46, the isolator 36 can include one sleeve disposed at either the first end 40 or second end 42. In one or more embodiments, the isolator 36 can have no sleeves (see, e.g., isolator 236 of hearing device 212 of FIG. 9).

The second end 30 of the front housing 24 can be connected to the first sleeve 44 of the isolator 36 using any suitable technique, e.g., bonding, adhering including adhesive bonding and adhesive tapes, welding, friction-fitting, snap fitting, etc. Further, the second end 34 of the rear housing 26 can be connected to the second sleeve 46 of the isolator 36 using any suitable technique, e.g., the same techniques described herein regarding connecting the front housing 24 to the first sleeve 44.

The body 38 of the isolator 36 can take any suitable shape and have any suitable dimensions. Further, the body 38 can include any suitable material, e.g., the same materials described herein regarding the front and rear housings 24, 26. In one or more embodiments, the body 38 of the isolator 36 includes elastomeric material, e.g., silicone, neoprene, isoprene, ethylene propylene diene monomer rubber (EPDM), etc. In one or more embodiments, the body 38 includes the same material as at least one of the front housing 24 or the rear housing 26. The body 38 can be manufactured using any suitable technique, e.g., molding, injection molding, 3D printing, die-casting, metal injection molding, sintering, stamping, casting, etc.

The body 38 of the isolator 36 can exhibit any desirable hardness value. In one or more embodiments, the hardness value of the body 38 is at least 20 durometer Shore 00. In one or more embodiments, the hardness value of the body 38 is no greater than 80 durometer Shore A. In one or more embodiments, the hardness value of the body 38 is at least 10 durometer Shore D. In one or more embodiments, the hardness value of the body 38 is no greater than 100 durometer Shore D. Further, the sleeves 44, 46 of the isolator 36 can exhibit any desirable hardness value. In one or more embodiments, the hardness value of each of the sleeves 44, 46 is at least 20 durometer Shore 00. In one or more embodiments, the hardness value of each of the sleeves 44, 46 is no greater than 80 durometer Shore A. In one or more embodiments, the hardness value of each of the sleeves 44, 46 is at least 10 durometer Shore D. In one or more embodiments, the hardness value of each of the sleeves 44, 46 is no greater than 100 durometer Shore D. In one or more embodiments, the hardness value of each of the sleeves 44, 46 of the isolator 36 is greater than the hardness value of the body 38 of the isolator 36. In one or more embodiments, the hardness value of each of the sleeves 44, 46 of the isolator 36 is less than the hardness value of the body 38 of the isolator 36. The first and second sleeves 44, 46 can have the same hardness value or different hardness values.

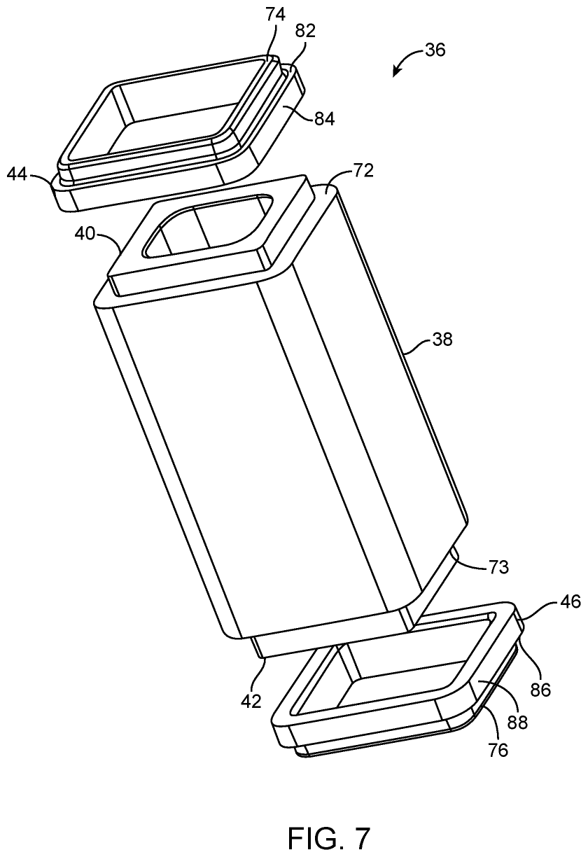

The first sleeve 44 of the isolator 36 can be connected to any suitable portion or portions of the body 38. For example, the isolator body 38 can include a ledge 72 (FIG. 7) disposed proximate to the first end 40, where the first sleeve 44 is disposed on or engaged with the ledge. Further, for example, the isolator body 38 can include a ledge 73 disposed proximate to the second end 42, wherein the second sleeve 46 is disposed on or engaged with the ledge. The first and second sleeves 44, 46 can be connected to the body 38 using any suitable technique, e.g., bonding, adhering including adhesive bonding and adhesive tapes, welding, friction-fitting, snap fitting, etc. In one or more embodiments, at least one of the first sleeve 44 or second sleeve 46 can be over-molded onto one or more portions of the body 38. Each of the sleeves 44, 46 can be a unitary component or include two or more portions.

The first sleeve 44 can include a rib 74 (FIG. 7) that extends in a direction away from the first end 40 of the body 38. The rib 74 can be adapted to be inserted into the second end 30 of the front housing 24. Further, the second sleeve 46 can include a rib 76 that extends in a direction away from the second end 42 of the body 38, where the rib is adapted to be inserted into the second end 34 of the rear housing 26. Each of the ribs 74, 76 can take any suitable shape. In one or more embodiments, the second end 30 of the front housing 24 can include a slot 78 (FIG. 3) that is adapted to receive the rib 74 of the first sleeve 44. Further, in one or more embodiments, the second end 34 of the rear housing 26 can include a slot 80 that is adapted to receive the rib 76 of the second sleeve 46.

The first sleeve 44 of the isolator 36 can also include a ledge 82 (FIG. 7) that extends from the rib 74 to a perimeter 84 of the first sleeve. Further, the second sleeve 46 can also include a ledge 86 that extends from the rib 76 to a perimeter 88 of the second sleeve. An end surface 90 (FIG. 3) of the second end 30 of the front housing 24 is adapted to engage the ledge 82 of the first sleeve 44 when the front housing is connected to the isolator 36. Further, an end surface 92 of the second end 34 of the rear housing 26 is adapted to engage the ledge 86 of the second sleeve 46 when the rear housing is connected to the isolator 36.

Although the isolator 36 is depicted as including the first and second sleeves 44, 46, in one or more embodiments, the isolator can include only the body 38 disposed between the front housing 24 and the rear housing 26. For example, FIG. 9 is a schematic cross-section view of another embodiment of a hearing device 212. All design considerations and possibilities described herein regarding the hearing device 12 of FIGS. 1-8 apply equally to the hearing device 212 of FIG. 9. The hearing device 212 includes an enclosure 222 that has a front housing 224 and a rear housing 226. The device 212 also includes an isolator 236 disposed between the front housing 224 and the rear housing 226. The isolator 236 includes a body 238.

One difference between the hearing device 212 of FIG. 9 and the hearing device 12 of FIGS. 1-8 is that a second end 230 of the front housing 224 and a second end 234 of the rear housing 226 are connected to the body 238 of the isolator 236 and not to a sleeve (e.g., first and second sleeves 44, 46). Any suitable technique can be utilized to connect the front and rear housings 224, 226 to the body 238. The isolator body 238 can take any suitable shape and have any suitable dimensions, e.g., the same shape and dimensions described herein regarding the isolator body 38 of isolator 36 of FIGS. 1-8. Further, the isolator body 238 can include any suitable material, e.g., the same materials described herein regarding the isolator body 38 of isolator 36.

Returning to FIG. 3, the first sensor 100 is disposed within the front housing 24. The first sensor 100 can be disposed in any suitable location within the front housing 24. In one or more embodiments, the first sensor 100 is disposed within the slot 54 disposed in the front housing 24.

The first sensor 100 can include any suitable sensor or sensors e.g., at least one of a temperature, optical, or tactile sensor. In one or more embodiments, the first sensor 100 is at least one microphone, e.g., a MEMS microphone, an electret condenser microphone, co-joined microphone sets, etc. The first sensor 100 is operatively connected to the opening 50 of the front housing 24 by the first sensor port 52. In embodiments where the first sensor 100 includes a microphone, the first sensor port 52 can be an acoustic port that acoustically connects the microphone and the opening 50 using any suitable technique. Further, the first sensor port 52 can take any suitable shape and have any suitable dimensions. In one or more embodiments, the first sensor 100 can include a manifold 95 that extends along the enclosure axis 2 and defines a portion of the acoustic port 48. In one or more embodiments, the first sensor port 52 can be nano-coated to resist debris and moisture ingress.

The second sensor 102 can also include any suitable sensor or sensors e.g., at least one of a temperature, optical, or tactile sensor. In one or more embodiments, the second sensor 102 is at least one microphone, e.g., a MEMS microphone, an electret condenser microphone, co-joined microphone sets, etc. In one or more embodiments, each of the first and second sensors 100, 102 includes a microphone.

The second sensor 102 is operatively connected to the opening 108 disposed adjacent to the first end 32 of the rear housing 26 by the second sensor port 106 that extends between the inlet 112 of the second sensor and the opening. In embodiments where the second sensor 102 includes a microphone, the second sensor port 106 can be an acoustic port that acoustically connects the microphone and the opening 108 using any suitable technique. Further, the second sensor port 106 can take any suitable shape and have any suitable dimensions. In one or more embodiments, the second sensor port 106 can be nano-coated to resist debris and moisture ingress.

The opening 108 of the second sensor port 106 can take any suitable shape and have any suitable dimensions. Further, the opening 108 can be disposed in any suitable portion or portions of the rear housing 26. As shown in FIG. 2, the opening 108 is disposed in a side surface 31 of the rear housing 26 and the first end 32 of the rear housing. In one or more embodiments, the opening 108 can be disposed entirely within the side surface 31 of the rear housing 26 or entirely within the first end 32 of the rear housing. In one or more embodiments, the opening 108 can extend through the rear housing 26 to one or more additional side surfaces of the rear housing.

The second sensor port 106 can be a vented port that remains acoustically open to an ambient environment of the hearing device 12. The hearing device 12 can include a valve that can at least partially occlude the second sensor port 106 such that acoustic energy from the ambient environment is at least partially obstructed from reaching the second sensor 102. Any suitable valve can be utilized to at least partially occlude the second sensor port 106. Further, the valve can be disposed in any suitable location relative to the second sensor port 106. In one or more embodiments, the valve can be disposed within the second sensor port 106. Such valve can be adapted to prevent ingress of fluid or debris into the second sensor port 106 and to an interior of the enclosure 22 of the hearing device 12.

Disposed at least partially within the body 38 of the isolator 36 is the receiver 104. The receiver 104 can be disposed in any suitable location within the body 38. In one or more embodiments, at least a portion of the receiver 104 can be disposed in the front housing 24 or the rear housing 26. In one or more embodiments, a first portion of the receiver 104 can be disposed in the front housing 24 and a second portion can be disposed in the rear housing 26.

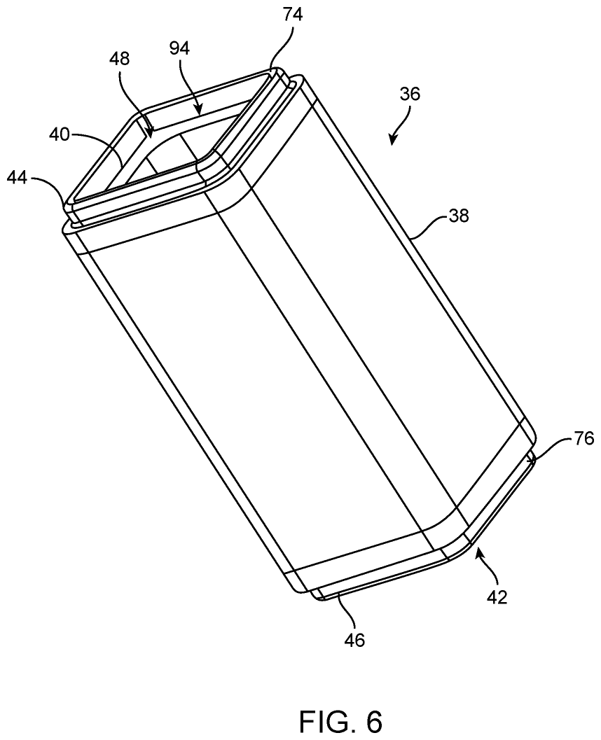

The receiver 104 can include any suitable receiver or receivers, e.g., a balanced armature speaker, dynamic driver speaker, piezo electric speaker, etc. The receiver 104 is acoustically connected to the opening 50 in the front housing 24 by the acoustic port 48 that extends through the isolator body 38 between the receiver and the opening. As can be seen in FIG. 6, the body 38 of the isolator 36 includes an opening 94 that forms a portion of the acoustic port 48. In one or more embodiments, the acoustic port 48 can be provided by a tube or other structure that is inserted through the opening 94 of the body 38 of the isolator 36. The acoustic port 48 can take any suitable shape and have any suitable dimensions.

As can be seen, e.g., in FIG. 3, an inlet 96 of the first sensor port 52 defines a plane 4 that is orthogonal to the enclosure axis 2. An outlet 98 of the acoustic port 48 can be any suitable distance from the plane 4. In one or more embodiments, the distance between the outlet 98 of the acoustic port 48 and the plane 4 is no greater than 1.2 mm as measured in a direction parallel to the enclosure axis 2. In one or more embodiments, the outlet 98 of the acoustic port 48 is parallel to the inlet 96 of the microphone port 52, i.e., in the same plane 4 as the inlet of the microphone port.

Returning to FIG. 1, the hearing module 14 can be adapted to be disposed between the ear and the skull of the wearer. The hearing module 14 includes the module housing 18 and electronic components 20 (FIG. 8) disposed within the module housing. The electronic components 20 of the hearing module 14 can include any suitable electronic component or circuitry, e.g., at least one of a controller, an integrated circuit, a power source, a microphone, or a speaker (i.e., receiver). In one or more embodiments, the electronic components 20 of the hearing module 14 include a controller 21 that is operatively connected to the hearing device 12 using any suitable technique, e.g., by the cable 16. In one or more embodiments, the electronic components 20 of the hearing module 14 can be electrically connected to the hearing device 12 by the cable 16. Further, in one or more embodiments, the electronic components 20 can be connected to the hearing device 12 by a wireless connection using any suitable wireless technique.

In one or more embodiments, the controller 21 of the hearing module 14 can be adapted to direct a noise canceling signal to the receiver 104 of the hearing device 12 that is based upon a noise signal received from the first sensor 100 of the hearing device. The receiver 104 is adapted to direct a noise canceling acoustic wave into the ear canal of the wearer that is based upon this noise canceling signal from the controller 21. Further, in one or more embodiments, the controller 21 can be adapted to determine the fit of the hearing device 12 in the ear canal of the wearer based on a feedback signal from the first sensor 100 of the hearing device 12 in response to a fit-test acoustic wave directed into the ear canal by the receiver 104. In one or more embodiments, the controller 21 is adapted to measure a pulse rate of the wearer based on a pulse signal received from the first sensor 100 of the hearing device 12. The pulse signal is based on a pulse detected by the first sensor 100 using any suitable technique, e.g., one or more of the techniques described in U.S. Patent Publication No. 2020/0268265 A1, entitled INTEGRATION OF SENSOR-BASED CARDIOVASCULAR MEASURES INTO PHYSICAL BENEFIT MEASURE RELATED TO HEARING INSTRUMENT USE. In one or more embodiments, the controller 21 can be adapted to detect the wearer's voice utilizing at least one of the first sensor 100 or second sensor 102 and provide a signal representative of such voice to the wearer using any suitable technique, e.g., one or more of the techniques described in U.S. Pat. No. 9,699,573, entitled HEARING ASSISTANCE SYSTEM WITH OWN VOICE DETECTION; and U.S. Pat. No. 9,042,586, entitled METHOD AND APPARATUS FOR OWN-VOICE SENSING IN A HEARING ASSISTANCE DEVICE. In one or more embodiments, the controller 21 is adapted to measure an occlusion value of the hearing device 12 in the ear canal based on an occlusion signal received from at least one of the first sensor 100 or second sensor 102 of the hearing device in response to an acoustic wave directed into the ear canal by the receiver 104 and detected by the first sensor using any suitable technique, e.g., one or more of the techniques described in U.S. Patent Application No. 63/286,780, entitled HEARING DEVICE AND METHOD OF USING SAME. In one or more embodiments, the controller 21 is adapted to detect one or more respiratory sounds of the wearer based on a respiratory signal received from at least one of the first sensor 100 or second sensor 102 using any suitable technique, e.g., one or more of the techniques described in U.S. Patent Application No. 63/295,071, entitled EAR-WEARABLE DEVICES AND METHODS FOR RESPIRATORY CONDITION DETECTION AND MONITORING. Further, in one or more embodiments, the controller 21 can be adapted to measure at least one of heartbeat variability, respiration rate, otoacoustic emissions, damage to the tympanic membrane, or canal tissue inflammation utilizing at least one of the first sensor 100 or second sensor 102. Further, in one or more embodiments, one or both of the first and second sensors 100, 102 along with the controller 21 can be utilized for feedback detection and suppression using any suitable technique.

The electronic components 20 of the hearing module 14 can further include one or more microphones. In embodiments where the first and second sensors 100, 102 are microphones, the microphone of the hearing module 14 can provide a three-microphone system that can provide enhanced direction performance of the hearing device system 10.

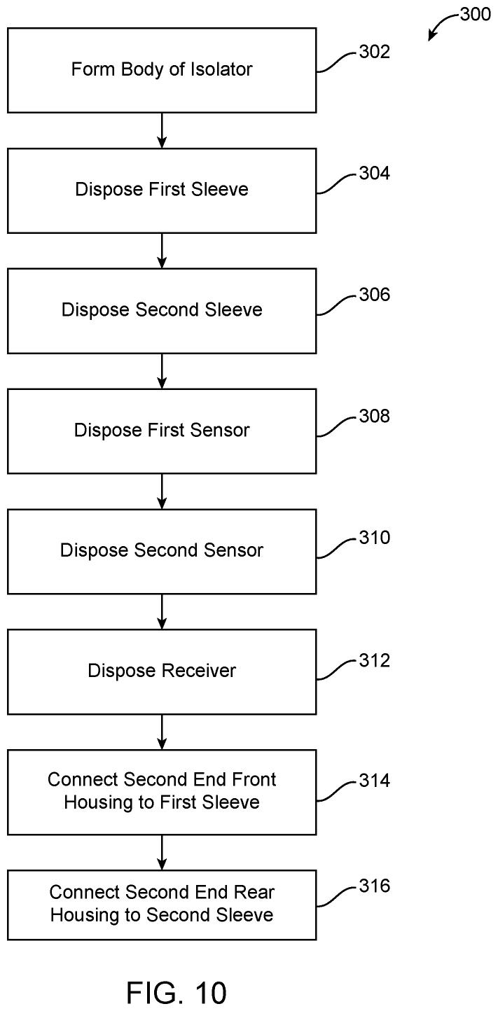

Any suitable technique can be utilized to form the various embodiments of hearing devices described herein. For example, FIG. 10 is a flowchart of one embodiment of a method 300 of forming the hearing device 12. Although described regarding the hearing device 12 of FIGS. 1-8, the method 300 can be utilized to form any suitable hearing device.

At 302, the body 38 of the isolator 36 can be formed using any suitable technique, e.g., molding. The first sleeve 44 can be disposed at the first end 40 the body 38 of the isolator 36 at 304 using any suitable technique, e.g., the first sleeve can be over-molded onto the body of the isolator. Although not shown, in one or more embodiments, the opening 94 can be disposed through the body 38 of the isolator 36 using any suitable technique. At 306, the second sleeve 46 can be disposed at the second end 42 of the body 38 of the isolator 36 using any suitable technique, e.g., the second sleeve can be over-molded onto the body of the isolator. In one or more embodiments, the first and second sleeves 44, 46 can be overmolded onto the body 38 of the isolator 36 simultaneously or in separate steps.

At 308, the first sensor 100 can be disposed within the front housing 24 (e.g., at least partially within the slot 54 formed in the inner surface 56 of the front housing) at 308 using any suitable technique. Further, at 310, the second sensor 102 can be disposed within the rear housing 26 using any suitable technique. At 312, the receiver 104 can be disposed at least partially within the body 38 of the isolator 36 using any suitable technique. The second end 30 of the front housing 24 can be connected to the first sleeve 44 of the isolator 36 at 314 using any suitable technique. For example, in one or more embodiments, the end surface 90 of the front housing 24 can be adhered to at least one of the rib 74 or the ledge of the first sleeve 44 using any suitable adhesive. Further, at 316, the second end 34 of the rear housing 26 can be connected to the second sleeve 46 of the isolator 36 using a suitable technique. For example, in one or more embodiments, the end surface 92 of the second end 34 of the rear housing 26 can be adhered to at least one of the rib 76 or the ledge 86 of the second sleeve 46 using any suitable adhesive.

All references and publications cited herein are expressly incorporated herein by reference in their entirety into this disclosure, except to the extent they may directly contradict this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below.

What is claimed is:

1. A hearing device comprising:
an enclosure extending along an enclosure axis and comprising:
a front housing that extends along the enclosure axis between a first end and a second end and comprising an opening disposed in the first end;
a rear housing that extends along the enclosure axis between a first end and a second end; and
an isolator disposed between the front housing and the rear housing, wherein the isolator comprises a body that extends along the enclosure axis between a first end and a second end, a first sleeve disposed at the first end of the body, and a second sleeve disposed at the second end of the body, wherein the second end of the front housing is connected to the first sleeve of the isolator and the second end of the rear housing is connected to the second sleeve of the isolator;
a first sensor disposed in the front housing;
a second sensor disposed in the rear housing; and
a receiver disposed at least partially within the body of the isolator.

2. The device of claim 1, further comprising an acoustic port that extends through the first end of the isolator body between the receiver and the opening disposed in the first end of the front housing, wherein the acoustic port acoustically connects the receiver to the opening.

3. The device of claim 2, further comprising a first sensor port that extends between the first sensor and the opening disposed in the first end of the front housing, wherein the first sensor port operatively connects the first sensor to the opening.

4. The device of claim 3, wherein an inlet of the first sensor port defines a plane that is orthogonal to the enclosure axis, wherein a distance between an outlet of the acoustic port and the plane is no greater than 1.2 mm as measured in a direction parallel to the enclosure axis.

5. The device of claim 3, wherein the first sensor further comprises a manifold that extends along the enclosure axis and defines a portion of the first sensor port.

6. The device of claim 3, further comprising a second sensor port that extends between an inlet of the second sensor and an opening disposed adjacent to the first end of the rear housing, wherein the second sensor port operatively connects the second sensor to the opening.

7. The device of claim 1, wherein the isolator body comprises a ledge disposed proximate to the first end, wherein the first sleeve is disposed on the ledge.

8. The device of claim 1, wherein the isolator body comprises an elastomeric material.

9. The device of claim 1, wherein the first sleeve of the isolator comprises a rib that extends in a direction away from the first end of the body, wherein the rib is adapted to be inserted into the second end of the front housing.

10. The device of claim 9, wherein the first sleeve of the isolator further comprises a ledge that extends from the rib to a perimeter of the first sleeve, wherein an end surface of the second end of the front housing is adapted to engage the ledge.

11. The device of claim 1, wherein the second sleeve of the isolator further comprises a rib that extends in a direction away from the second end of the body, wherein the rib is adapted to be inserted into the second end of the rear housing.

12. The device of claim 11, wherein the second sleeve of the isolator further comprises a ledge that extends from the rib to a perimeter of the second sleeve, wherein an end surface of the second end of the rear housing is adapted to engage the ledge.

13. The device of claim 1, wherein at least one of the first sensor or the second sensor comprises a microphone.

14. A hearing device system comprising:
a hearing device comprising:
an enclosure extending along an enclosure axis and comprising:
a front housing that extends along the enclosure axis between a first end and a second end and comprises an opening disposed in the first end;
a rear housing that extends along the enclosure axis between a first end and a second end; and
an isolator disposed between the front housing and the rear housing, wherein the isolator comprises a body that extends along the enclosure axis between a first end and a second end, a first sleeve disposed at the first end of the body, and a second sleeve disposed at the second end of the body, wherein the second end of the front housing is connected to the first sleeve of the isolator and the second end of the rear housing is connected to the second sleeve of the isolator;
a first sensor disposed in the front housing;
a second sensor disposed in the rear housing; and
a receiver disposed at least partially within the body of the isolator;
a hearing module adapted to be disposed between an ear and a skull of a wearer, wherein the hearing module comprises a module housing and electronic components disposed within the module housing; and
a cable that connects the hearing device to the hearing module.

15. The system of claim 14, wherein the electronic components of the hearing module comprise a controller that is operatively connected to the hearing device.

16. The system of claim 15, wherein the controller is adapted to direct a noise canceling signal to the receiver of the hearing device that is based upon a noise signal received from the first sensor of the hearing device, wherein the receiver is adapted to direct a noise canceling acoustic wave into an ear canal of the wearer of the hearing device that is based upon the noise canceling signal from the controller.

17. The system of claim 15, wherein the controller is adapted to determine a fit of the hearing device in an ear canal of the wearer based upon a feedback signal from the first sensor of the hearing device in response to a fit-test acoustic wave directed into the ear canal by the receiver.

18. The system of claim 15, wherein the controller is adapted to measure a pulse rate of the wearer based upon a pulse signal received from the first sensor of the hearing device, wherein the pulse signal is based upon a pulse detected by the first sensor.

19. The system of claim 15, wherein the controller is adapted to measure an occlusion value of the hearing device in an ear canal of the wearer based upon an occlusion signal received from the first sensor of the hearing device in response to an acoustic wave directed into the ear canal by the receiver and detected by the first sensor.

20. The system of claim 14, wherein the electronic components of the hearing module comprise a microphone, wherein each of the first sensor and second sensor comprises a microphone.

21. A method comprising:
forming a body of an isolator;
disposing a first sleeve at a first end of the body;
disposing a second sleeve at a second end of the body;
disposing a first sensor within a front housing, wherein the front housing comprises a first end, a second end, and an opening disposed in the first end;
disposing a second sensor within a rear housing, wherein the rear housing comprises a first end and a second end;
disposing a receiver at least partially within the body of the isolator;
connecting the second end of the front housing to the first sleeve; and
connecting the second end of the rear housing to the second sleeve, wherein the front housing, the isolator, and the rear housing define an enclosure that extends along an enclosure axis.
